(19) United States
(12) Patent Application Publication
(10) Pub. No.: US 2024/0382095 A1
(43) Pub. Date: Nov. 21, 2024

(54) REAL-TIME ADAPTATION OF A PERSONALIZED HEART MODEL

(71) Applicant: SORIN CRM SAS, CLAMART (FR)

(72) Inventors: JEREMY BERCOFF, PARIS (FR); SERGE CAZEAU, PARIS (FR)

(21) Appl. No.: 18/688,750

(22) PCT Filed: Sep. 3, 2021
(86) PCT No.: PCT/EP2021/074368

§ 371 Date: Mar. 1, 2024

(51) Int. Cl. A61B 5/02; A61B 5/0215; A61B 5/349; A61B 5/352; A61B 8/08; A61M 60/178; A61M 60/196; A61M 60/216; A61M 60/403; A61M 60/515; A61N 1/365; A61N 1/39

(52) U.S. Cl. CPC A61B 5/02028; A61B 5/0215; A61B 5/349; A61B 5/352; A61B 8/0883; A61B 8/488; A61M 60/178; A61M 60/196; A61M 60/216; A61M 60/403; A61M 60/515; A61N 1/36585; A61N 1/3956

(57) ABSTRACT

This invention relates to a method and system for determining and/or tracking a heart function of a patient. wherein the heart function is estimated by combining information representative of an electrical, mechanical and/or hemodynamic heart function received from imaging, therapy and/or diagnosis systems or a patient database with real-time information representative of an electrical, mechanical and/or hemodynamic heart function received from a measuring device attached or implanted to the patient. The information and the real-time information from can be associated to update an evaluation of the heart function of the patient in real-time and thereby improve the heart function and possibly optimize its efficiency.

### FIELD OF THE INVENTION

The invention relates to a heart function monitoring system for determining and/or tracking a heart function of a patient.

### BACKGROUND OF THE INVENTION

The heart is a complex organ precisely controlled by the interplay of electrical and mechanical fields. It consists of four chambers (left and right atria and left and right ventricles) connected by four valves, which act in concert to regulate its filling, ejection, and overall pump function. A coordinated opening and closing of these four valves regulates the filling of the chambers, while the interplay of electrical and mechanical effects controls their proper ejection. Disturbed valvular opening, stenosis, disturbed closing, regurgitation, disturbed electrical signals, arrhythmias, and reduced mechanical function, heart failure, can have devastating physiological consequences. Irrespective of its nature and initial location, heart disease almost always progresses to affect the entire heart, and eventually impairs the electrical and mechanical function of all four chambers.

To understand the fundamental pathologies of different forms of heart disease and optimize their treatment options, it is critical to model the entire heart as a whole rather than studying the diseased subsystem in complete isolation. Modelling the interplay between electrical excitation and mechanical contraction provides better insight into the complex heart functions and holds the potential to improve treatment for people affected by heart disease.

While numerous computational models exist to study either the electrical or the mechanical response of its individual chambers, the integrative electromechanical response of the whole heart remains very complex Creating whole heart models remains challenging for various reasons including that various structures with different functions coexist, mechanical and hemodynamic constraints interplay one with the others and potential modifications of shape and pressure regimens modify further anatomy. Furthermore, modelling atria is different from ventricles and modelling the right heart is different from modelling the left heart. In addition, the atria are typically entangled and their geometry can be quite complex. This not only complicates image segmentation, but also atrial discretization and meshing.

Much effort to develop digital models of the heart has been undertaken in recent years, as described e.g. in Baillargeon, Brian, et al.: “The living heart project: a robust and integrative simulator for human heart function”, European Journal of Mechanics-A/Solids 48 (2014), pp 38-47. These models have mainly been used for product design. Their use in medical therapy is limited by their theoretical nature, not being representative of the real heart of a patient, and the slowness of computer calculations visualizing the effect of the simulations that can be carried out.

Among the therapies that can improve the heart function in patients with heart failure, cardiac resynchronization therapy (CRT) is a minimally invasive and effective solution that improves patients' functional capacity, increases their life expectance and reduces the rate of hospitalization. However, as described in “2013 ESC Guidelines on cardiac pacing and cardiac resynchronization therapy: the Task Force on cardiac pacing and resynchronization therapy of the European Society of Cardiology (ESC). Developed in collaboration with the European Heart Rhythm Association (EHRA)”, Europace (2013), 15, 1070-1118, CRT is faced with a non-response ratio of 30 to 35% of presently eligible patients. This non-response ratio may be caused by at least one of a weak predictive value of the ECG signal as single tool (especially because of low-performance cardiac dyssynchrony identification, an implantation of stimulation probes that do not take into account particular characteristics of the patient's heart, and a resynchronization strategy optimization selected according to wrong or poor criteria (e.g., delay AV/VV) and without personalization and adaptation.

Initiatives for using digital heart models to estimate the heart function as close as possible are described in S. Cazeau et al.: “Multisite stimulation for correction of cardiac asynchrony”, Heart 2000; 84, pp 579-81, S. Cazeau et al.: “Statistical ranking of electromechanical dysynchrony parameters for CRT”, Open Heart 2019; 6, and M. Sermesant et al.: “Patient-specific electromechanical models of the heart for the prediction of pacing acute effects in CRT: A preliminary clinical validation”; Med. Image Anal. (2011), doi:10.1016/j.media.2011.07.003.

However, such heart models are valid at a given point in time only, because underlying information and hypotheses are derived at a given time of patient life. The evolution of the state of health or therapy of the patient will change the evaluated situation and therefore can induce an erroneous assessment and tracking.

### SUMMARY OF THE INVENTION

It is an object of the present invention to provide a method and apparatus for real-time determination and tracking of the quality of the heart function and in particular its degree of dyssynchrony.

This object is achieved by an apparatus as claimed in claim 1, a real-time heart function quality tracking system as claimed in claim 12, a method as claimed in claim 14, and a computer program product as claimed in claim 17.

Accordingly, real-time tracking of the heart function is proposed by relying on a continuous personalization of a digital heart model. This can be achieved by associating all or a portion of the parameters of the heart model with measurements made by a medical device constantly carried by the patient reflecting at all times its health condition.

According to first aspect, an apparatus for determining and/or tracking a heart function of a patient is provided, wherein the apparatus is configured to estimate the heart function by combining information representative of an electrical, mechanical and/or hemodynamic heart function received from imaging, therapy and/or diagnosis systems or a patient database with real-time information representative of an electrical, mechanical and/or hemodynamic heart function received from a measuring device attached or implanted to the patient.

According to a second aspect, a method of determining and/or tracking a heart function of a patient is provided, wherein the method comprises estimating the heart function by combining information representative of an electrical, mechanical and/or hemodynamic heart function received from imaging, therapy and/or diagnosis systems or a patient database with real-time information representative of an electrical, mechanical and/or hemodynamic heart function received from a measuring device attached or implanted to the patient

According to a third aspect, a real-time heart function quality tracking system is provided, which comprises an apparatus of the first aspect and further comprises:
an information gathering system for retrieving the information representative of the electrical, mechanical and/or hemodynamic heart function from the imaging, therapy and/or diagnosis systems or the patient database;
a diagnostic or therapy system permanently worn by the patient to measure electrical, mechanical and/or hemodynamic signals from the heart of the patient; and
a user interface usable for a diagnostic support by a physician or for a support in delivery of a therapy and its optimization.

According to a fourth aspect, a computer program product is provided, which comprises code means for producing the steps of the above method of the second aspect when run on a computer device.

According to a first option of any one of the first to fourth aspects, the information representative of the electrical heart function may be derived from an electrocardiogram signal, the information representative of the mechanical heart function may be derived from a tissue Ultrasound Doppler mode or strain signal, and the information representative of the hemodynamic heart function may be derived from a Ultrasound Doppler spectrum of a ventricle.

According to a second option which may be combined with the first option or any one of the first to fourth aspects, the real-time information representative of the electrical heart function may be derived from an electrogram signal and the real-time information representative of the hemodynamic heart function may be derived from a signal reflecting an intracardiac parameter (such as intracardiac pressure or intracardiac volume or another hemodynamic signal) of a bio-impedance type.

According to a third option which may be combined with any one of the first and second options or any one of the first to fourth aspects, the real-time information representative of the mechanical heart function may be derived from a signal reflecting cardiac vibration.

According to a fourth option which may be combined with any one of the first to third options or any one of the first to fourth aspects, a personalized electromechanical model of the heart of the patient may be built using the information representative of an electrical, mechanical and/or hemodynamic heart function.

According to a fifth option which may be combined with any one of the first to fourth options or any one of the first to fourth aspects, at least some parameters of a left pre-ejection interval (LPEI), a right pre-ejection interval (RPEI), a diastolic ventricular filling time (DFT), a diastolic filling duration reported to heart rate (DFT %), a duration of contraction of the septum (Sept), a diastolic contraction at the septal level (DCsept), an isovolumic relaxation time (IsovolRT), a duration of contraction of a lateral level (LLW), a diastolic contraction of the lateral wall, (DClat), an overlap of the Septum and/or the left lateral wall contractions with the onset of the next filling phase of the heart (OvlapSept and OvlapLLW), meaning that these segments exhibit diastolic contractions (DCsept and DClat), a beat-to-beat interval (RR), a septal left lateral wall (Sept-LLW), an onset time of the next E wave (QRS-E), an interventricular delay (IVD), a systole duration (SD), a left ventricular ejection time (LVET), an isovolumic contraction time (IsovolCT), a ratio of the area of the flow to left atrial area (MVR/LA), and a ratio LPEI/LVET may be extracted from the information representative of an electrical, mechanical and/or hemodynamic heart function.

According to a sixth option which may be combined with any one of the first to fifth options or any one of the first to fourth aspects, the at least some parameters may be extracted from the information representative of an electrical, mechanical and/or hemodynamic heart function by an artificial intelligence method.

According to a seventh option which may be combined with any one of the first to sixth options or any one of the first to fourth aspects, the electromechanical model of the heart may be personalized using the at least some parameters and physiological data of the patient and statistical data on the pathology retrieved from the patient database.

According to an eighth option which may be combined with any one of the first to seventh options or any one of the first to fourth aspects, a cardiac resynchronization therapy, CRT, device or a cardiac assist pump may be controlled to improve the estimated cardiac function.

According to a ninth option which may be combined with any one of the first to eighth options or any one of the first to fourth aspects, stimulation delays applied to electrodes of the CRT device may be selected to improve the estimated cardiac function.

According to a tenth option which may be combined with any one of the first to ninth options or any one of the first to fourth aspects, the heart function may be estimated based on a quantification of cardiac dyssynchrony.

According to an eleventh option which may be combined with any one of the first to tenth options or any one of the first to fourth aspects, the diagnostic or therapy system of the third aspect may comprise a measuring device configured as a holter-type diagnostic device or a cardiac resynchronization therapy device for measuring the electrical, mechanical and/or hemodynamic signals, and a therapy device configured as a type of left ventricular assist device, LVAD, pump, the cardiac resynchronization therapy device, or an artificial heart for assisting the heart function of the patient.

According to a twelfth option which may be combined with any one of the first to eleventh options or any one of the first to fourth aspects, an imaging procedure may be applied to extract the information representative of the electrical, mechanical and/or hemodynamic heart function; a personalized electro-mechanical model of the heart of the patient may be built using the information extracted during the imaging procedure; a measurement in permanent or intermittent contact with the patient may be performed to obtain the real-time information representative of the electrical, mechanical and/or hemodynamic heart function; and the information and the real-time information may be associated to update the estimation of the cardiac function of the patient in real-time.

According to a thirteenth option which may be combined with any one of the first to twelfth options or any one of the first to fourth aspects, a location of electrodes of the CRT device may be selected to improve the estimated cardiac function.

It is noted that the above apparatus may be implemented based on discrete hardware circuitries with discrete hardware components, integrated chips, or arrangements of chip modules, or based on signal processing devices or chips controlled by software routines or programs stored in memories, written on a computer readable media, or downloaded from a network, such as the Internet.

It shall be understood that the apparatus of claim 1, the system of claim 12, the method of claim 14, and the computer program product of claim 17 may have similar and/or identical preferred embodiments, in particular, as defined in the dependent claims.

It shall be understood that a preferred embodiment of the invention can also be any combination of the dependent claims or above embodiments with the respective independent claim.

These and other aspects of the invention will be apparent from and elucidated with reference to the embodiments described hereinafter.

### BRIEF DESCRIPTION OF THE DRAWINGS

In the following drawings:

FIG. 1 shows schematically a block diagram of a real-time heart function quality tracking system according to various embodiments;

FIG. 2 shows a flow diagram of a real-time heart function quality tracking method according to various embodiments;

FIG. 3 shows schematically various diagrams with waveforms of measured signals representative of electrical activity and hemodynamics of a monitored heart; and

FIG. 4 shows a flow diagram of a more detailed exemplary real-time heart function quality tracking method according to an embodiment.

### DETAILED DESCRIPTION OF EMBODIMENTS

Various embodiments of the present invention are now described based on a heart function quality tracking system in which electrical and mechanical signals derived from at least one of imaging, therapy and diagnosis are combined with electrical and mechanical signals measured at the heart.

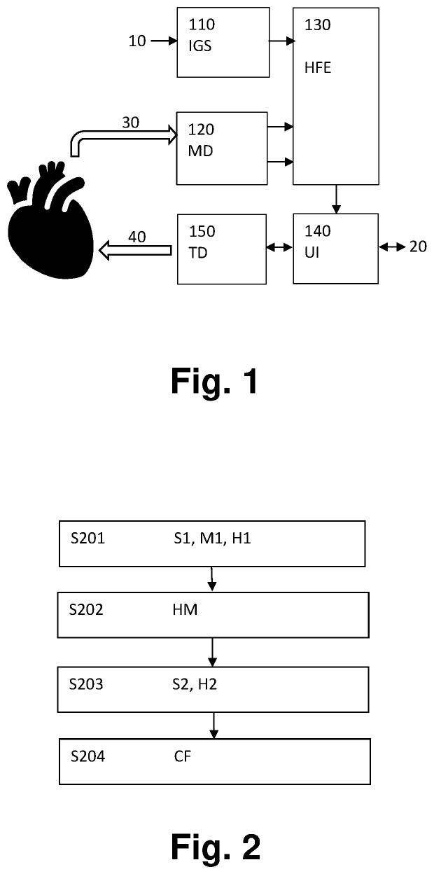

FIG. 1 shows schematically a block diagram of a real-time heart function quality tracking system according to various embodiments.

It is noted that—throughout the present disclosure—the structure and/or function of blocks with identical reference numbers that have been described before are not described again, unless an additional specific functionality is involved. Moreover, only those structural elements and functions are shown, which are useful to understand the embodiments. Other structural elements and functions are omitted for brevity reasons.

The real-time heart function quality tracking system of FIG. 1 can be implemented as an integrated (centralized) or distributed (decentralized) system comprising at least one apparatus or device for determining and/or tracking a heart function including an information gathering device or system (IGS) 110, which may be a computer system, for retrieving and processing non-real-time patient information 10 (e.g., at least one of electrical (S1), mechanical (M1) and hemodynamic (H1) signals) obtained from imaging, therapy, or diagnosis systems or a patient database.

Additionally, a diagnostic or therapy system or device which may be permanently or intermittently worn (e.g., implanted or attached) by the patient comprises a therapy device (TD) 150, such as a heart stimulator or the like, for applying therapeutic signals (e.g., stimulating signals) 40 and a measuring device (MD) 120 for measuring at least one of electrical (S2), mechanical (M2) and hemodynamic (H2) signals 30 from the heart. The measuring device 120 and/or the therapy device 150 may be a holter type diagnostic device (implantable or not), a pacemaker or implantable cardioverter/defibrillator (ICD) device, a cardiac resynchronization therapy (CRT) device, or a therapy device for assisting the heart function, e.g. of a left ventricular assist device (LVAD) pump or an artificial heart.

The measuring device 120 may be configured to measure an electrogram (EGM) signal as a real-time electrical signal and a signal of the bio-impedance type reflecting the intracardiac pressure as a real-time hemodynamic signal. Furthermore, a signal representative of the electrical function of the heart may be an ECG signal, a signal representative of the mechanical function of the heart may be a tissue Ultrasound Doppler mode or strain or a mechanical signal reflecting cardiac vibration measured e.g. by an accelerometer or piezoelectric type sensor of the measuring device 120, and a signal representative of the hemodynamic function of the heart may be a Ultrasound Doppler spectrum of the ventricle.

Furthermore, a user interface (UI) 140 may be used for inputting and/or outputting therapeutic and/or diagnostic control information 20 from/to a physician or for support in delivery of a therapy and its later optimization.

Moreover, a controller or signal processor 130 is provided for performing a heart function estimation (HFE) estimation based on a combination of non-real-time electrical and/or mechanical signals received from the information gathering system 110 and the real-time mechanical and/or electrical signals 30 measured by the measuring device 120 of the diagnostic or therapy system or device worn by the patient. The non-real-time electrical and/or mechanical signals may be received, derived or calculated by the information gathering system 110 based on the non-real-time patient information 10.

FIG. 2 shows a flow diagram of a real-time heart function quality tracking method according to various embodiments, which may be implemented by the controller 130 of FIG. 1 by executing respective instructions stored in a memory (not shown) of the real-time heart function quality tracking system.

In step S201, non-real-time electrical and/or mechanical signals are received from the information gathering system 110, which comprise at least one of a signal S1 representative of the electrical function of the individual patient's heart, a signal M1 representative of the mechanical function of the individual patient's heart and a signal H1 representative of the hemodynamic function of the individual patient's heart. The signals S1, M1 and H1 may be obtained by an imaging process, such as cardiac ultrasound or the like.

In step S202, a modeling process is performed for building a personalized electromechanical model of the heart of the patient using the received information (i.e., signals S1, M1 and/or H1) extracted during the imaging process. The modeling may include extracting electromechanical parameters of the heart from the signal(s) S1, M1 and/or H1 representative of the electrical and mechanical functions of the individual patient's heart.

As an example, potential correlations between electromechanical parameters (e.g., atrioventricular, interventricular and intraventricular parameters obtained from a dyssynchrony model) and their ability to describe dyssynchrony and their potential use in resynchrony has been evaluated in Serge Cazeau et al.: “Statistical ranking of electromechanical dyssynchrony parameters for CRT”, Open Heart 2019. More specifically, 455 sets of 18 parameters of the dyssynchrony model obtained in 91 patients submitted to various pacing configurations were evaluated two-by-two using a Pearson correlation test and then by groups according to their ability to describe dyssynchrony, using the Column selection method of machine learning. As a result, a powerful parameter is duration of septal contraction, which unfortunately describes only 25% of dyssynchrony, imposing the association of several parameters. Furthermore, the best groups of 3, 4 and ≥8 variables describe 59%, 73% and almost 100% of dyssynchrony, respectively. Left pre-ejection interval is highly and significantly correlated to a maximum of other variables, and its decrease is associated with the favorable evolution of all other correlated parameters. Increase in filling duration and decrease in duration of septum to lateral wall contraction difference are not associated with the favorable evolution of other parameters. It can be concluded that no single electromechanical parameter alone can fully describe dyssynchrony. Although the 18-parameter dyssynchrony model can be simplified, it still requires at least 4-8 parameters. Decrease in left pre-ejection interval favorably drives resynchrony in a maximum of other parameters.

In the present modeling case, electromechanical parameters to be extracted may include at least some of a left pre-ejection interval (LPEI), a right pre-ejection interval (RPEI), a diastolic ventricular filling time (DFT), a diastolic filling duration reported to heart rate (DFT %), a duration of contraction of the septum (Sept), a diastolic contraction at the septal level (DCsept), an isovolumic relaxation time (IsovolRT), a duration of contraction of a lateral level (LLW), a diastolic contraction of the lateral wall (DClat), an overlap of the Septum and/or the left lateral wall with the onset of the next filling phase of the heart (OvlapSept and OvlapLLW), meaning that these segments exhibit diastolic contractions (DCsept and DClat), a beat-to-beat interval (RR), a septal left lateral wall (Sept-LLW), an onset time of the next E wave (QRS-E), an interventricular delay (IVD), a systole duration (SD), a left ventricular ejection time (LVET), an isovolumic contraction time (IsovolCT), a ratio of the area of the flow to left atrial area (MVR/LA), and a ratio LPEI/LVET.

In an example, the above electromechanical parameters may be extracted from the electrical and/or mechanical signals S1, M1 and/or H1 by processing the respective signal to isolate and analyze an electrocardiogram (ECG) signal to determine the onset of each QRS wave and other ones of the above parameters, or by applying artificial intelligence (AI) methods, as described later.

The extracted electromechanical parameters are then used to personalize a heart model. This may be achieved by using all or part of these parameters as well as with access to a heterogeneous database including physiological data of the patient and statistical data on the pathology.

Then, in step S203, a measurement step by a measuring device (e.g., measuring device 120) in permanent or intermittent contact with the patient is performed to obtain real-time electrical, mechanical and hemodynamic signals S2, M2, H2, e.g., at least a second electrical signal S2 representative of the electrical function and a second hemodynamic signal H2 representative of the hemodynamic function H2 of the heart.

Finally, in step S204, the heart function of the individual patient's heart is estimated by associating the extracted parameters and signals representative of electrical, mechanical and/or hemodynamic functions respectively derived from the non-real-time electrical, mechanical and/or hemodynamic signals (e.g. S1, M1, H1) received in step S201 and the real-time electrical, mechanical and/or hemodynamic signals (e.g. S2, M2, H2) measured in step S203 are associated to update the evaluation (modeling) of the heart function of the patient in step S202 in real-time. Thereby, the heart function can be improved or updated and its efficiency can be optimized.

In an example, the modeling process in step S202 and the estimation of the heart function in step S204 may be made by AI methods using all or at least some (e.g. signals S1, S2) of the non-real-time signals S1, M1, H1 and the real-time signals S2 and H2 and optionally physiological data from the patient.

In an example, AI learning based on a convolutional neural network (CNN) may be used for analyzing patient's imaging information (e.g., image slices) or other non-real-time signals and extracting at least some of the above targeted parameters. A CNN is a regularized version of a multilayer neural network, which takes advantage of a hierarchical pattern in data and assembles patterns of increasing complexity using smaller and simpler patterns embossed in its filters. CNNs use relatively little pre-processing compared to other image classification algorithms. This means that the network learns to optimize the filters (or kernels) through automated learning. This independence from prior knowledge and human intervention in feature extraction is a major advantage.

Another example for extracting at least some of the above targeted parameters based on pattern detection using machine learning may be the so-called K-means clustering method which is a method of vector quantization that aims at partitioning N observations into K clusters in which each observation belongs to the cluster with the nearest mean (cluster centers or cluster centroid), serving as a prototype of the cluster. This results in a partitioning of the data space into cells. In the present case, the parameter K (i.e., number of clusters) may vary from 3 to 20 depending on the resolution and the quality of the patterns to be detected. This method can be used to detect outliers based on their plotted distance from the closest cluster. K-means clustering method involves the formation of multiple clusters of data points each with a mean value. In the present case, such data points may be one-dimensional data points (e.g., clustering of time series of data patterns of the electrical and/or mechanical signals S1, M1 and/or H1 based on e.g. value ranges of the data patterns) or two-dimensional data points (e.g., clustering in a two-dimensional plane of data patterns of a first parameter characteristic vs. a second parameter characteristic) or three-dimensional data points (e.g., clustering in a three-dimensional space of data patterns of a first parameter characteristic vs. a second parameter characteristic vs. a third parameter characteristic). Typical data patterns will then form cluster(s), while anomalous data patterns will be located at a larger distance from the cluster of typical data patterns. Objects within a cluster have the closest mean value. Any object with a threshold value greater than the nearest cluster mean value is identified as an outlier. A step-by-step method used in K-means clustering may involve calculating a mean value of each cluster, setting an initial threshold value, determining the distance of each data point from the mean value during a testing process (learning phase), and identifying the cluster that is nearest to the test data point. If a distance value is more than a predetermined threshold value is then mark it as an outlier.

The above list of electromechanical parameters or a portion P thereof forms an R\hat{ }P dimension space where each log parameter adds a given point in this space. A multi-dimensioned unsupervised clustering approach (K-means) can then be used to cluster the log-points into K clusters (K is determined by the desired resolution on the data for the specific analytic and ranges from 1 to 10 with a typical default of 6 for most cases).

If a supervised learning algorithm is used for heart function estimation (modeling) and/or parameter extraction requires the existence of a labeled dataset that contains both normal and anomalous data points. Examples of supervised methods include anomaly detection using neural networks, Bayesian networks, and the K-nearest neighbors (or k-NN) method. Supervised anomaly detection provides a better rate of anomaly detection in the output signal thanks to their ability to encode any interdependency between variables and including previous data in any predictive model.

In another example, the AI-based heart function estimation and/or parameter extraction may be implemented as an unsupervised learning algorithm that allows raw, unlabeled data to be used to train the heart function estimation and/or parameter extraction with little or no human involvement during the learning process. In unsupervised learning, the heart function estimation simply receives the electrical or mechanical signals to extract parameters and estimate the personalized heart model or function. Thus, no training data with manual labeling are required. These methods are based on a statistical assumption that most of the inflowing data are normal and only a minor percentage would be anomalous data. These methods also estimate that any malicious data would be different statistically from normal data. Some of the unsupervised methods include the above K-means method, autoencoders, and hypothesis-based analysis. As a result, an improved AI-based parameter extraction and/or heart function estimation can be achieved for the real-time heart function quality tracking system or method, which is trained by a self-learning process and is thus readily available with little or no human involvement.

Thus, the above AI-based approaches with AI-based algorithms enable to predict and extract at least some of the above targeted parameters, so that better decisions can be made on the fly to create a better personalized heart model and adapt it to the actual situation or condition of the patient's heart. Due to the learning capability of heart function estimation, better adaptation of the personalized heart model to the individual characteristic of the patient's heart can be achieved.

In a further example, the estimation of the heart function may be based on a quantification of cardiac dyssynchrony. This quantification may as well be based on the above AI-based approaches.

In another example, the estimation of the heart function may be performed before and after applying a therapy to the patient by a CRT apparatus or cardiac assist pump. Based thereon, the functions of the CRT apparatus or cardiac assist pump may be adjusted to improve the estimated heart function. This may be achieved by placing the electrodes of the CRT apparatus to improve the estimated heart function and/or by selecting stimulation delays applied to the electrodes of the CRT apparatus to improve the estimated heart function.

The information extracted from each of the above process steps S201 to S204 (electromechanical signals, model parameters, indication of therapy, optimization of therapy) may be provided to a physician using fully digital and instantaneous treatment.

In an exemplary embodiment, a patient may be indicated for cardiac ultrasound imaging. The cardiologist performs the cardiac ultrasound imaging with at least four cuts, which may be apical view of four chambers plus spectral doppler, apical view of five chambers plus spectral doppler, sax view (short axis) plus spectral doppler, and optionally plax view (parasternal long axis) M Mode, 4-channel color M Mode view or strain view.

Furthermore, a trace ECG may be measured by an echo device and displayed on all views.

The echo system may then store and export the above views (e.g., in Dicom format) to be directly or later uploaded to the real-time heart function quality tracking system (which may be implemented in computer cloud) dedicated to the analysis of heart function.

The real-time heart function quality tracking system then extracts at least some of the above listed electromechanical parameters from the echo images, e.g., by processing the conventional echo signal to isolate and analyze the ECG signal to determine the onset of each QRS, or by applying a CNN-type artificial intelligence method for analyzing the echographic image slices and extracting the targeted parameters.

Of course, other algorithms mentioned above can be used for parameter extraction and/or heart function estimation.

A digital model of the heart (e.g., the dyssynchrony model described above) is then customized or personalized based on the extracted parameter set and an dyssynchrony score may be calculated. This score may then be sent back to the cardiologist in real time via a dedicated user interface (e.g., the user interface 140 of FIG. 1).

The patient is then indicated for the implantation of a therapy device of the CRT type allowing measurement of electrical, mechanical and/or hemodynamic signals (S2, M2 and H2).

Once the device is implanted, the electrical (S2) and hemostatic (H2) and possibly mechanical (M2) signals can be measured periodically by the device to obtain real-time patient information.

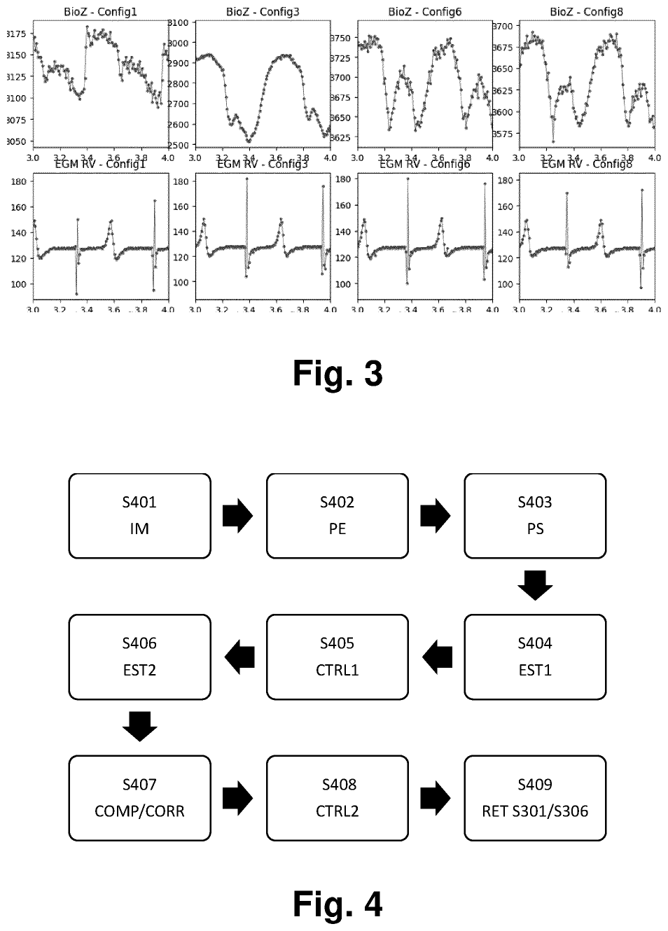

FIG. 3 shows schematically various diagrams with waveforms of measured signals representative of electrical activity and hemodynamics of a monitored heart.

These signals are an impedance cardiography (BioZ) (upper waveform diagrams of FIG. 3) representative of cardiac hemodynamics for different configurations (Config1 to Config8) and an EGM (lower waveform diagrams of FIG. 3) for a signal representative of electrical activity for the different configurations (Config1 to Config8).

The impedance cardiography is a non-invasive measurement of the function of the heart and blood vessels, which may include cardiac output (e.g., blood volume pumped by the heart with each cycle), systemic vascular resistance (e.g., resistance to blood flow offered by blood vessels) and fluid status (e.g., total amount of fluids in the body). Furthermore, blood pressure may be determined by the balance between two factors, e.g., the volume of blood pumped by the heart per minute or the cardiac output and the ability of the blood vessels to dilate to accommodate the needs of your body, or systemic vascular resistance.

From these two signals a subset of the above electromechanical parameters (such as for example the LPEI) are extracted in real time.

In another example, at least some of the real-time parameters may be used to feed the personalized heart model and/or update the asynchrony estimation.

A comparison of the LPEI and some other parameters measured by imaging and by the therapy device allows “emancipation” from imaging. Therefore, automatic comparison with preimplant data gives continuous evaluation of resynchrony delivery. The measuring probes can be located at positions minimizing the value of the LPEI parameters and stimulation delays can be optimized in the same way.

The selected set or all electromechanical parameters listed above are monitored in real time by the implant (daily or several times a day). As soon as a significant variation is detected by the real-time heart function quality tracking system or method for the implant indicating an excessive dyssynchrony, at least one of the following two actions can be carried out:
a self-optimization of the therapy (optimization of stimulation delays can be initiated to reduce dyssynchrony;
an alert can be transmitted to the physician via the real-time heart function quality tracking system (e.g., cloud computer) so that the physician can summon the patient and adjust the therapy if necessary.

FIG. 4 shows a flow diagram of a more detailed exemplary real-time heart function quality tracking method according to an embodiment.

In an initial step S401, an echocardiographic (ECG) imaging (IM) of the patient is performed. Then, in step S402, electromechanical parameters are extracted (PE) from the ECG images or slices. Thereafter, in step S403, a patient's digital heart model is personalized (PS) using the extracted and input electromechanical parameters. In the following step S404, the state or quality of the heart function is estimated (EST1), such as for example, the rate of dyssynchrony measured at a time T0 during an imaging process. In the next step S405, an implanted or attached therapy device (e.g., the therapy device 150 of FIG. 1) is controlled (CTRL1) by a physician or automatically by a controller (e.g., the heart function estimation controller 130 of FIG. 1). In subsequent step S406, electromechanical parameters are estimated (EST2) in real time via an implanted or attached measuring device (e.g., the measuring device 120 of FIG. 1) at a current time T1.

In step S407, the two sets of parameters (i.e., non-real-time and real-time parameters) are compared and/or correlated (COM/CORR) to assess the evolution of the heart function of the patient. In response thereto, the control settings of the implanted or attached therapy device are updated (CTRL2) by the physician or automatically by the controller in step S408.

Finally, in step S409, the procedure returns to step S406 to initiate a new real-time measurement or to S401 to initiate a new imaging process.

To summarize, a method and system for determining and/or tracking a heart function of a patient have been described, wherein the heart function is estimated by combining information representative of an electrical, mechanical and/or hemodynamic heart function received from imaging, therapy and/or diagnosis systems or a patient database with real-time information representative of an electrical, mechanical and/or hemodynamic heart function received from a measuring device attached or implanted to the patient. The information and the real-time information from can be associated to update an evaluation of the heart function of the patient in real-time and thereby improve the heart function and possibly optimize its efficiency.

While the invention has been illustrated and described in detail in the drawings and foregoing description, such illustration and description are to be considered illustrative or exemplary and not restrictive. The invention is not limited to the disclosed embodiments. The proposed extraction and estimation procedures can be used for any medical devices for treatment of human beings and animals. In particular, the invention can be applied to any kind of measuring devices for measuring real-time electrical, mechanical and/or hemodynamical information and any kind of imaging, therapy, diagnosis or patient data from which electrical, mechanical and/or hemodynamic information can be derived for extracting at least one of the above listed electromechanical parameters.

Other variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfil the functions of several items recited in the claims. The mere fact that certain measures are recited in mutually different dependent claims does not indicate that a combination of these measures cannot be used to advantage. The foregoing description details certain embodiments of the invention. It will be appreciated, however, that no matter how detailed the foregoing appears in the text, the invention may be practiced in many ways, and is therefore not limited to the embodiments disclosed. It should be noted that the use of particular terminology when describing certain features or aspects of the invention should not be taken to imply that the terminology is being re-defined herein to be restricted to include any specific characteristics of the features or aspects of the invention with which that terminology is associated.

The described operations or procedures like those indicated in FIGS. 2 and 4 can be implemented as program code means of a computer program and/or as dedicated hardware of the receiver devices or transceiver devices, respectively. The computer program may be stored and/or distributed on a suitable medium, such as an optical storage medium or a solid-state medium, supplied together with or as part of other hardware, but may also be distributed in other forms, such as via the Internet or other wired or wireless telecommunication systems.

1. An apparatus for determining and/or tracking a heart function of a patient, wherein the apparatus is configured to estimate the heart function by combining information representative of an electrical, mechanical and/or hemodynamic heart function, received from imaging, therapy and/or diagnosis systems or a patient database, with real-time information representative of an electrical, mechanical and/or hemodynamic heart function received from a measuring device attached or implanted to the patient.

2. The apparatus of claim 1, wherein the information representative of the electrical heart function is derived from an electrocardiogram signal, the information representative of the mechanical heart function is derived from a tissue Ultrasound Doppler mode or strain signal, and the information representative of the hemodynamic heart function is derived from an Ultrasound Doppler spectrum of a ventricle.

3. The apparatus of claim 1 or 2, wherein the real-time information representative of the electrical heart function is derived from an electrogram signal and the real-time information representative of the hemodynamic heart function is derived from a signal reflecting an intracardiac pressure of a bio-impedance type.

4. The apparatus of any one of claims 1 to 3, wherein the real-time information representative of the mechanical heart function is derived from a signal reflecting cardiac vibration.

5. The apparatus of any one of the preceding claims, wherein the apparatus is configured to build a personalized electromechanical model of the heart of the patient using the information representative of an electrical, mechanical and/or hemodynamic heart function.

6. The apparatus of any of the preceding claims, wherein the apparatus is configured to extract at least some parameters of a left pre-ejection interval, LPEI, a right pre-ejection interval, RPEI, a diastolic ventricular filling time, DFT, a diastolic filling duration reported to heart rate, DFT %, a septal level, a duration of contraction of the septum, Sept, a diastolic contraction at the septal level, DCsept, an isovolumic relaxation time, IsovolRT, a duration of contraction of a lateral level, LLW, a diastolic contraction of the lateral wall, DClat, an overlap of the Septum and/or the left lateral wall contractions with the onset of the next filling phase of the heart, OvlapSept and OvlapLLW, meaning that these segments exhibit diastolic contractions, DCsept and DClat, a beat-to-beat interval, RR, a septal left lateral wall, Sept-LLW, an onset time of the next E wave, QRS-E, an interventricular delay, IVD, a systole duration, SD, a left ventricular ejection time, LVET, an isovolumic contraction time, IsovolCT, a ratio of the area of the flow to left atrial area, MVR/LA, and a ratio LPEI/LVET from the information representative of an electrical, mechanical and/or hemodynamic heart function.

7. The apparatus of claim 6, wherein the apparatus is configured to extract the at least some parameters from the information representative of an electrical, mechanical and/or hemodynamic heart function by an artificial intelligence method.

8. The apparatus of claim 6 or 7, wherein the apparatus is configured to personalize the electromechanical model of the heart using the at least some parameters and physiological data of the patient and statistical data on the pathology retrieved from the patient database.

9. The apparatus of any one of the preceding claims, wherein the apparatus is configured to control a cardiac resynchronization therapy, CRT, device or a cardiac assist pump to improve the estimated cardiac function.

10. The apparatus of claim 8, wherein the apparatus is configured to select stimulation delays applied to electrodes of the CRT device to improve the estimated cardiac function.

11. The apparatus of any one of the preceding claims, wherein the apparatus is configured to estimate the heart function based on a quantification of cardiac dyssynchrony.

12. A real-time heart function quality tracking system comprising an apparatus (130) of any of the preceding claims and further comprising:
an information gathering system (110) for retrieving the information representative of the electrical, mechanical and/or hemodynamic heart function from the imaging, therapy and/or diagnosis systems or the patient database;
a diagnostic or therapy system (120, 150) permanently worn by the patient to measure electrical, mechanical and/or hemodynamic signals from the heart of the patient; and
a user interface (140) usable for a diagnostic support by a physician or for a support in delivery of a therapy and its optimization.

13. The system of claim 12, wherein the diagnostic or therapy system comprises a measuring device (120) configured as a holter-type diagnostic device or a cardiac resynchronization therapy device for measuring the electrical, mechanical and/or hemodynamic signals, and a therapy device (150) configured as a type of left ventricular assist device, LVAD, pump, a cardiac resynchronization therapy device, or an artificial heart for assisting the heart function of the patient.

14. A method of determining and/or tracking a heart function of a patient, wherein the method comprises estimating the heart function by combining information representative of an electrical, mechanical and/or hemodynamic heart function, received from imaging, therapy and/or diagnosis systems or a patient database, with real-time information representative of an electrical, mechanical and/or hemodynamic heart function received from a measuring device attached or implanted to the patient.

15. The method of claim 14, further comprising:
applying an imaging procedure to extract the information representative of the electrical, mechanical and/or hemodynamic heart function;
building a personalized electro-mechanical model of the heart of the patient using the information extracted during the imaging procedure;
performing a measurement in permanent or intermittent contact with the patient to obtain the real-time information representative of the electrical, mechanical and/or hemodynamic heart function; and
associating the information and the real-time information to update the estimation of the cardiac function of the patient in real-time.

16. The method of claim 14 or 15, further comprising selecting a location of electrodes of a CRT device to improve the estimated cardiac function.

17. A computer program product comprising code means for producing the steps of any one of claims 14 to 16 when run on a computer device.
